(19) United States
(12) Patent Application Publication
(10) Pub. No.: US 2007/0287180 A1
(43) Pub. Date: Dec. 13, 2007

(54) Linear Dna Fragment For Markerless Deletion, Novel Strain Having Inhibited Formation Of Biofilm And Preparation Method Thereof

(76) Inventors: Sun-Chang Kim, Dacjeon (KR); Choong-Hoon Lee, Gyeonggi-do (KR); Bo-Ram Lim, Gyeongsangbuk-do (KR); Bong-Hyun Sung, Gyeongsangnam-do (KR); Byung-Jo Yu, Daejeon (KR); Won-Sik Lee, Chungcheongbuk-do (KR); Jun-Hyoung Lee, Daegu (KR); Sang-Hee Lee, Gyeongsangbuk-do (KR)

Correspondence Address: LATHROP & CLARK LLP, 740 REGENT STREET SUITE 400, P.O. BOX 1507, MADISON, WI, 537011507, US

(21) Appl. No.: 11/574,484

(22) PCT Filed: Sep. 1, 2005
(86) PCT No.: PCT/KR05/02898

§ 371 Date: Feb. 28, 2007

(30) Foreign Application Priority Data
Sep. 2, 2004 (KR) 10-2004-0070017

(51) Int. Cl. C12N 15/70; C07H 21/04; C12N 1/21

(52) U.S. Cl. 435/455; 435/252.33; 536/23.1

(57) ABSTRACT

The present invention relates to Escherichia coli variants that have increased antibiotics susceptibility, diffusion efficiency, and transformation efficiency. The variants can minimize the problems caused by biofilm formation such as increased resistance to antibiotics, decreased solute diffusion efficiency, and lowered transformation efficiency. According to the present invention, when selecting genetically-modified E. coli variants, not only a lesser amount of antibiotics is required when selecting desirable variants, but also the reduction of selection efficiency caused by biofilm formation by strains other than the variants to be selected, thus decreasing exhibiting resistance to antibiotics, can be avoided. Additionally, in the process of materials production, the amount of secreted products could be increased due to the increased solute diffusion efficiency. Furthermore, the increased transformation efficiency makes the mass production of useful materials easier.

### TECHNICAL FIELD

The present invention relates to Escherchia coli variants that have increased antibiotics susceptibility, diffusion efficiency, and transformation efficiency. The variants can minimize the problems caused by biofilm formation such as increased resistance to antibiotics, decreased solute diffusion efficiency, and lowered transformation efficiency.

### BACKGROUND ART

Biofilms are formed by clusters of microorganisms that adhere to a biotic or abiotic surface. A biofilm formation blocks,the diffusion of solutes in the environment. Since diffusion of antibiotics, which are one type of the solutes, is also blocked, the microorganisms inside of the biofilm exhibit resistance to the antibiotics. Therefore, a strain could exhibit undesirable resistance to the antibiotics. In other words, a strain that should be removed by killing or inhibiting its growth can continue to survive and grow. As a result, when preparing different types of variants, the parent cell, rather than the desirable variants, could survive, thus reduces the selection efficiency.

Additionally, in the process of materials production, the amount of secreted products could be reduced due to biofilm formation. When preparing a transformant that incorporates a foreign gene, the transfer or the incorporation of the foreign gene can be inhibited due to biofilms, and thus its transformation efficiency is decreased. Particularly, increased biofilm formation by high density cell culture and long culture time in the bioindustrial processes causes severe problem for production of useful materials.

Two major approaches have been used for the inhibition of biofilm formation in bioindustry: one approach is the addition of biofilm inhibiting agents to the growth media and the other is the development of new materials on which bacteria cannot adhere. A successful case, however, has yet been reported due to the limitations such as an environmental pollution problem and high cost.

Therefore, an object of the present invention is to provide Eschelichia coil variants that can enhance antibiotics susceptibility, solute diffusion efficiency, and transformation efficiency by inhibiting biofilm formation. These biofilm-deficient E. coli variants can be obtained by deleting of genes that are related to the formation of biofilm.

Another object of the present invention is to provide a method to construct E. coli variants that exhibit increased antibiotics susceptibility, solute diffusion efficiency, and transformation efficiency, comprising a markerless deletion wherein a selective marker is not remained after the deletion of biofilm-related gene.

### DETAILED DESCRIPTION OF THE INVENTION

The present invention is related to E. coli variants that have increased antibiotics susceptibility, increased solute diffusion efficiency and higher transformation efficiency by deleting at least one of three biofilm-related genes in E. coli.

In the case of E. coli, curli, type I pili, and colonic acid are required for biofilm formation. It has been reported that curli and type I pili are required for connecting the microorganisms or adhering to surfaces and colanic acid contributes to the biofilm architecture. In other words, when two E. coli are connected to each other using the curli and type I pili, colanic acid fills up the space between the two E. coli to form biofilms.

A biofilm-deficient E. coli variant that has increased antibiotic susceptibility, solute diffusion efficiency, and transformation efficiency was constructed by deleting csg operon and/or fim operon and/or wca gene clusters that are related to generation of curli, type I pili and colanic acid, respectively.

In the present specification, an “Escherichia coli (E. coli) variant” is defined as gene variants of E. coli that inhibit biofilm formation. The above-mentioned variants includes, for example, an E. coli variant, that has increased antibiotic susceptibility, solute diffusion efficiency and transformation efficiency can be constructed by deleting at least one of the wca gene clusters, fim operon and csg operon.

In addition, a “biofilm” is a cluster of microorganisms that lives on a surface of a biotic or abiotic material. The inhibition of biofilm formation can be detected by an adherence property test, which is explained in detail later.

Moreover, “increased antibiotic susceptibility” is the ability to kill more microorganisms with a smaller amount of the antibiotics. For example, antibiotics susceptibility can be measured by using ampicillin, streptomycin, and rifampicin, which are explained in detail later.

Moreover, “increased solute diffusion efficiency” is the ability to diffuse a larger amount of solutes in a given amount of time. For example, solute diffusion efficiency can be measured by a method that detects the amount of activity related to lipase in a medium, which is explained in detail later.

Furthermore, “higher transformation efficiency” can be measured by the calcium chloride-heat shock transformation method that is explained in detail later.

Csg operon (csgDEFGBA, b1037˜b1043, Blattner, F. R., Plunkett, G. III, Bloch, C. A., Perna, N. T., Burland, V., Riley, M., Collado-Vides, J., Glasner, J. D., Rode, C. K., Mayhew, G. F., Gregor, J., Davis, N. W., Kirkpatrick, H. A., Goeden, M. A., Rose, D. J., Mau, B. and Shao, Y Science 277, 1453, 1997) are related to curli biosynthesis and and transfer of the synthesized curli to the outside of the cell, fim operon (fimBEAICDEF, b4312˜b4320, Blattner, F. R., Plunkett, G. III, Bloch, C. A., Perna, N. T., Burland, V., Riley, M., Collado-Vides, J., Glasner, J. D., Rode, C. K., Mayhew, G. F., Gregor, J., Davis, N. W., Kirkpatrick, H. A., Goeden, M. A., Rose, D. J., Mau, B. and Shao, Y. Science 277, 1453, 1997) are related to type I pili biosynthesis and transfer of the synthesized type I pili to the outside of the cell, wca gene clusters (wza wzb wzc wcaABCDEF gmd wcaGHI manC manB wcaJ wzx wcaKL, b2044˜b2062, Blattner, F. R., Plunkett, G. III, Bloch, C. A., Perna, N. T, Burland, V., Riley, M., Collado-Vides, J., Glasner, J. D., Rode, C. K., Mayhew, G. F., Gregor, J., Davis, N. W., Kirkpatrick, H. A., Goeden, M. A., Rose, D. J., Mau, B. and Shao, Y. Science 277, 1453, 1997) are related to colanic acid biosynthesis and transfer of the synthesized colonic acid to the outside of the cell.

As an example of the present invention, the antibiotics susceptibility of E. coli DEB31, which has deleted all of the three genes, to ampicillin was increased about 25-fold; to streptomycin was about 4-fold; and to rifampicin was about 5-fold. Concerning the secretion of lipase, the diffusion efficiency was increased about 11%, and as to the transformation using pUC19 plasmid vector, the transformation efficiency was shown to be increased more than 20-fold.

The following is an experiment method used to obtain the present invention.

First, the E. coli variants eliminated with the above-mentioned genes can be obtained by the following steps:
(1) a step for preparing a linear DNA fragment containing a selectable marker, a sacB gene (SEQ ID NO:31), I-Scel restriction enzyme recognition site (SEQ ID NO: 32), and. homology arms that are homologous with a genomic regions of a microorganism;
(2) a step for replacing the linear DNA fragment prepared in step (1) with a specific region of the chromosome of the microorganism;
(3) a step for eliminating selectable marker by introducing I-Scel restriction enzyme expression vector and then incubating in a selective medium culture containing sucrose; and optionally
(4) a step for continuously deleting specific genomic regions of the variants by repeating the above steps (2) and (3).

The linear DNA fragment includes a selectable marker; homology arms A and C of 500 base pairs (bp) that are needed in λ-Red recombination; the homology arm B and I-SceI restriction enzyme recognition sites that are necessary to delete the selectable marker; and sacB gene to confirm the markerless deletion of the target regions (Refer to FIG. 1).

As a selectable marker, an antibiotics resistant gene can be used, and since the E. coli variant, which contains an antibiotic resistant gene inserted into a chromosome, exhibit resistance to the pertinent antibiotics, such resistance can be used to select the desirable variants from other types of variants.

A homology arm refers to a region homologous to a genomic region that is to be deleted, and such regions are represented as homology arms A, B and C according to the gene sequences and Figures. About 500 bp of the homology arms A and C are generated by PCR, thereby locating at the both ends of the linear DNA fragment. They are involved in A-Red recombination for inserting the linear DNA fragment into the microorganism. About 500 bp of the homology arm B connected to either of the homology arm A or C is obtained by PCR. It is involved in homologous recombination for deleting a selectable marker (refer to FIG. 2). When the genomic region B is adjacent to the genomic region C on the chromosome, the homology arm B is connected to the homology arm A on the linear DNA fragment. In this case, the homology arms A, B and C may be dependent on the genomic regions of a microorganism that are to be deleted.

Moreover, the I-SceI restriction enzyme recognition site (SEQ ID NO: 32) consists of 18 base pairs, and is adjacent to the homology arm B, which is involved in the homologous recombination when eliminating the selectable marker. Since the I-SceI restriction enzyme recognition site does not exist in the E. coil chromosome, the linear DNA fragment that is used for the gene deletion should contain the I-SceI restriction enzyme recognition site.

Since about 500 bp of homology arms are used at the above step, more specific recombination can occur, and thus, there is an advantage of a more precise deletion.

The linear DNA fragment can be transferred to a microorganism strain by using a standard electroporation method. The target region on the chromosome was replaced with the linear DNA fragment through the λ-Red recombination between the homology arms A and C located on the both ends of the target region and linear DNA fragment. The E. coli variant replaced with the linear DNA fragment was selected in a medium containing a type of antibiotics corresponding to the antibiotics resistant gene on the linear DNA fragment.

A vector for expression of I-SceI restriction enzyme was transformed into a selected variant. Then, expressed I-SceI induced homologous recombination between duplicants of the homology arm B on the chromosome to eliminate the selectable marker from the variant. The examples of the present invention use plasmid pST98AS (SEQ ID No: 33, ACCESSION AF170483, Posfai G. Kolisnychenko V, Berecski Z. Blattner F R. 1999. Markerless gene replacement in Escherichia coli stimulated by a double-strand break in the chromosome Nucleic Acids Res 27 (22), 4409-15) for the expression of I-SceI. Then, the microorganism variants were selected on the medium containing sucrose. Generally, sacB gene encodes levansucrase. The levansucrase hydrolyzes sucrose to glucose and fructose and synthesizes fructose-polymer, levan. Since the levan is toxic to cell, the microorganism that has the sacB gene cannot survive in a medium containing sucrose. Therefore, the variants that have deleted the selectable marker and the sacB gene by homologous recombination can be selected in a medium containing sucrose.

Moreover, specific gene regions can be deleted by repeating steps (2) and (3).

The method for eliminating the chromosomal region is shown in FIG. 2. In FIG. 2, a represents a linear DNA fragment containing a selectable marker, a sacB gene, an I-SceI restriction enzyme, recognizing site and the homology arms A, B and C; b represents a part of the parent chromosome desired to be deleted; c represents a chromosome replaced with the linear DNA fragment at a deletion target region by λ-Red recombination; d represents the homologous recombination between the duplicated region Bs on the chromosome by using I-SceI restriction enzyme, and e represents the chromosome that have deleted specific target region without remaining of any foreign marker.

The adherence of the variants that have deleted at least one of the wca gene clusters, fim operon and csg operon using the method mentioned above was measured using a method developed by Dorel (Dorel C. et al., FEMS Microbiology Letters, 178, 169, 1999). In other words, 24-well polystyrene plates containing 2 ml of LB medium were inoculated with the variants and were incubated for 48 hours to allow for biofilm formation. Each well's upper layer was removed to be mixed with those that were washed with LB twice to consider it as a planktonic cell. The biofilm cells attached to the well were obtained by using 1 ml of LB by pipetting. The concentration of planktonic cells and that of biolfilm cells were measured by plate counting method. In order to compare the results in numbers, an adherence percentage of the biofilm cells that were growing on the surface of the well was calculated by dividing the concentration of biofilm cells by the sum of the concentrations of the planktonic cells and biofilm cells.

The antibiotics susceptibility of the variants was measured using the modified technique developed by Whiteley (Whiteley M., Nature, 413, 860, 2001). In other words, 96-well polystyrene plates containing 0.2 ml of LB culture were inoculated with the variants to be incubated for 24 hours to allow biofilm formation. Each well was added with various concentrations of antibiotics ranging from 0.25 μg to 64 μg and were incubated for 10 hours. The number of living cells was measured by plate counting method.

The diffusion efficiency of lipase of variants was measured using the technique developed by Ahn (Ahn J. H., J. Bacteriol, 181, 1847, 1999). According to an example of the present invention, pHOPE (Ahn J. H., J. Bacteriol, 181, 1847, 1999) expression vector that contains genes required for production of lipase and pABC-ACYC (Ahn J. H., J. Bacteriol, 181, 1847, 1999) expression vector were used. The expression vectors were delivered to cells using a commonly-used electroporation. A 250 ml Erlenmeyer flask containing 50 ml of LB medium was inoculated and then cultivated. Then, after a set period of time, 1 ml of culture was harvested every predetermined period of time and was centrifuged at 13,000 rpm for 10 minutes to measure the activity of lipase at the emission wavelength of 405 nm. For the activity of lipase, the amount of lipase that can degrade 1 μ mol of p-nitrophenyl palmitate per min was defined as one unit.

The transformation efficiency of the variants was measured using a modified calcium chloride-heat shock transformation method developed by Molchanova (Molchanova, E. S., Genetika, 19, 375, 1983). A plasmid vector, pUC19 (New England Biolabs, Beverly, Mass.), was transferred to a wild type E. coli and the biofilm formation-inhibited E. coli DEB31 of the present invention. Competent cells of the variant that were placed on ice were added with plasmids vectors of various concentrations from 1 ng to 100 ng. After 30 minutes, heat-shock was applied for 90 minutes at 42° C. before adding 800 ml of LB plate, and then cells were allowed to stand for one hour at 37° C. The number of living variants was confirmed after spreading the cells on a LB plate containing ampicillin and incubating it for 16 hours at. 37° C.

### BRIEF DESCRIPTION OF DRAWINGS

FIG. 1 represents a linear DNA fragment used to delete a region of an E. coli chromosome.

FIG. 2 shows the steps of deleting a specific gene of an E. coli using the linear DNA fragment.

FIG. 3 shows the process of PCR to construct the linear DNA fragment.

FIG. 4 shows the results of the confirmation of the deletion of biofilm-related genes by measuring the adherence percentages of the E. coli variants of the present invention.

FIG. 5 shows the susceptibility of the E. coli variants to ampicillin according to the present invention.

FIG. 6 shows the susceptibility of the E. coli variants to streptomycin according to the present invention.

FIG. 7 shows the susceptibility of the E. coli variants to rifampicin according to the present invention.

FIG. 8 is a graph showing the solute diffusion efficiency of the E. coli variants according to the present invention.

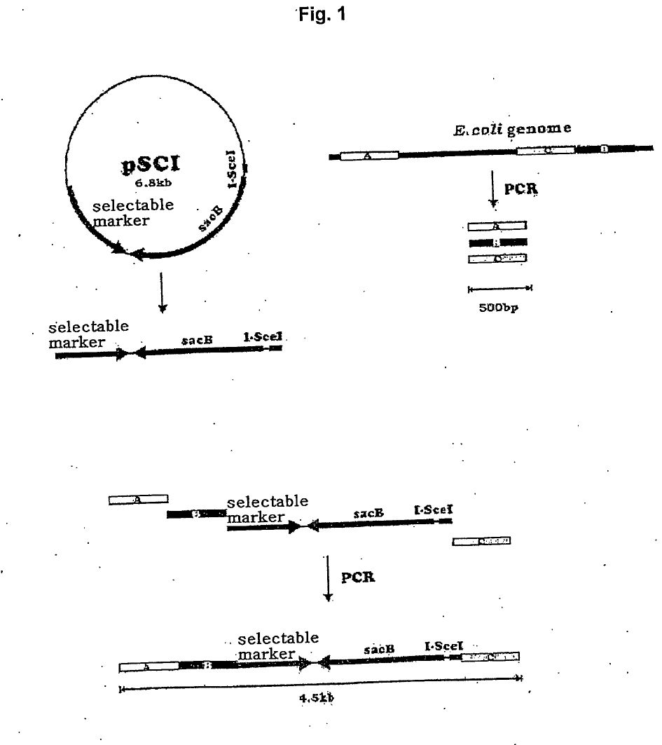

### EXAMPLES

The present invention will be further illustrated by the following examples. It will be apparent to those having conventional knowledge in the field that these examples-are given only to explain the present invention more clearly, but the invention is not limited to the examples given.

### Example 1

### Construction of Biofilm-Deficient E. coli Variants

E. coli DEB11, DEB12 and DEB13 are obtained by deleting csg operon, fim operon, and wca gene clusters from E. coli K-12 MG1655 (Professor Roe, Jung-Hye from Department of Microbiology, Seoul National University), respectively; each of the DEB21, DEB22, and DEB23 is obtained by deleting at least two genes of the csg operon, fim operon, and wca gene clusters; and E. coli variant DEB31 is obtained by deleting all of the csg operon, fim operon and wca gene clusters.

### 1-1. Preparation of csg Gene (csqDEFGBA)-Deleted E. coli DEB11

First, Cm^{R} gene (SEQ ID NO: 34) of pSG76C vector (Posfai G, Kolisnychenko V., Bereczki Z., Blattner F R. 1999. Markerless gene replacement in Escherichia coli stimulated by a double-strand break in the chromosome Nucleic Acids Res 27 (22), 4409-15) was digested with two restriction enzymes KpnI and BamHI (New England Biolabs, Beverly, Mass.) and cloned into the KpnI and BamHI site of pST76K vector (Posfai G. Kolisnychenko V., Bereczki Z., Blattner F R. 1999. Markerless gene replacement in Escherichia coli stimulated by a double-strand break in the chromosome Nucleic Acids Res 27 (22), 4409-15) containing I-SceI restriction enzyme recognition site (SEQ ID NO: 32). Then, the sacB gene (SEQ ID NO:31) from pDELTA vector (GIBCOBRL-DELETION FACTORY SYSTEM VERSION 2) was digested with restriction enzyme BamHI (New England Biolabs, Beverly, Mass.) and cloned into the plasmid with Cm^{R} gene and I-SceI recognition site. The constructed plasmid was designated as pSCI.

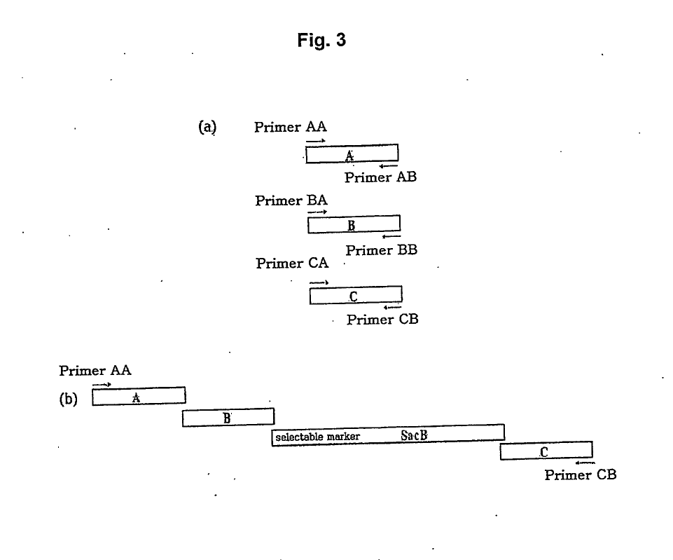

Subsequently, the homology arms A (SEQ ID NO: 7) and C (SEQ ID NO: 9), which are homologous to the regions at the both ends of target region (csgDEFGBA), were generated by recombinant PCR. The homology arms A and C are joined at the both ends of the vector to be involved in the λ-Red recombination. Moreover, the homology arm B (SEQ ID NO: 8) involved in eliminating the selectable marker by homologous recombination was generated and connected to one of homology arm of linear DNA by recombinant PCR. After positioning the homology arms A and C at both ends of the vector and positioning the homology arm B at one end, a linear DNA fragment (SEQ ID NO: 10) was generated by recombinant PCR. FIG. 3 (a) represents the process of PCR, and the following are the primers that were used in the process.

Primer AA:

(SEQ ID NO: 1)

5′-ggtgactggaaactggtgtta-3′
Primer AB:

(SEQ ID NO: 2)

5′-attatcggttatgaaagcaac-3′
Primer BA:

(SEQ ID NO: 3)

5′-gttgctttcataaccfataataccattattcctgaagtcactct-3′
Primer BB:

(SEQ ID No: 4)

5′-attaatttcgataagccagatcagttcatttctacgggtgatga-3′
Primer CA:

(SEQ ID NO: 5)

5′-gagtcgacctgcaggcatgcattgcagcaatcgtattct-3′
Primer CB:

(SEQ ID NO: 6)

5′-taaaggttatctgactggaaa-3′

The amplified DNAs were isolated using the Nucleogene Gel-Extraction KIT (Nucleongen, Gyeonggi-do, Korea).

The prepared linear DNA fragments were transferred to E. coli MG1655 strains (Roe, Jung-Hye from Department of Microbiology, Seoul National University) by using the standard eletroporation method [Bio-RAD, Bacterial electro-transformation and Plus Controller Instruction Manual Cat. No 165-2098; Thompson, J R, et al. An improved protocol for the preparation of yeast cells for transformation by electroporation. Yeast 14, 565-571 (1998); Grant, S G, et al. Differential plasmid rescue from transgenic mouse DNAs into Escherichia coli methyllation-restriction mutants. Proc. Natl. Acad. Sci. USA 87, 4645-4649 (1990)]. Since the linear DNA fragment contains two homology arms A and C that are homologous to the chromosomal region, the genomic region between the two homology arms is replaced with the linear DNA fragment. Since the E. coli variants containing the replaced the linear DNA fragments exhibit resistance to chloramphenicol due to the existence of Cm^{R} gene in the linear DNA fragments, they were selected on LB plate containing chloramphenicol.

The I-SceI expression vector, pST98AS (ACCESSION AF170483, Posfai G. Kolisnychenko V, Bereczki Z, Blattner F R. 1999. Markerless gene replacement in Escherichia coli stimulated by a double-strand break in the chromosome Nucleic Acids Res 27 (22), 4409-15) was introduced into the E. coli variants that have csg operon replaced with the selectable markers.

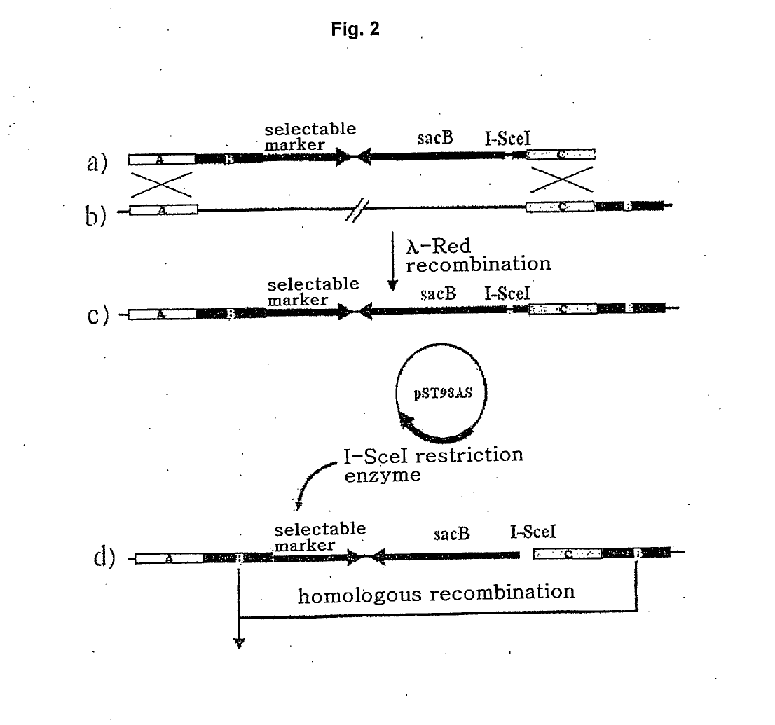

Since the transcription of I-SceI restriction enzyme in pST98AS can be controlled by tetracycline promoter, the restriction enzyme I-SceI was expressed by incubating on a plate containing chlortetracycline. During this process, the absence of sacB gene on the chromosome of the E. coli was tested by adding sucrose to the plate. Since a specific region of the chromosome containing selectable marker and sacB gene was deleted by homologous recombination between homology arm B duplicants induced by expressed I-SceI. Therefore, the variants can be selected in a medium containing sucrose. (Refer to FIG. 2). The deletion of csgDEFGBA genes from the selected variants was confirmed by PCR. The variant was named DEB11.

### 1-2. Preparation of DEB12 E. coli Variant that has Deleted fim Operon (fimBEAICDFGH)

Using the same method described in Example 1.1, the homology arms A (SEQ ID NO: 17), B (SEQ ID NO: 18), C (SEQ ID NO: 19) and linear DNA fragment (SEQ ID NO: 20) were prepared by using following primers, and the E. coli variant that has deleted fimBEAICDFGH genes was prepared, and thus was named DEB12.

Primer AA:

(SEQ ID NO: 11)

5′-caatctcatggcgtaagct-3′
Primer AB:

(SEQ ID NO: 12)

5′-gatttcactatgggtcagga-3′
Primer BA:

(SEQ ID NO: 13)

5′-cctgacccatagtgaaatcgtctgggattaacggcaa-3′
Primer BB:

(SEQ ID NO: 14)

5′-attaatttcgataagccagatctagatccagcaactggtca-3′
Primer CA:

(SEQ ID NO: 15)

5′-gagtcgacctgcaggcatgcccggaaaccattacagact-3′
Primer CB:

(SEQ ID NO: 16)

5′-ccgtgttattcgctggaa-3′

### 1-3. Preparation of DEB13 E. coli Variant that has Deleted wca Gene Clusters (wza wzb wzc wcaABCDEF gmd wcaGHI manC manB wcaJ wzx wcaKL)

Using the same method described in Example 1-1, the homology arms A(SEQ ID NO: 27), B (SEQ ID NO: 28), C (SEQ ID NO: 29) and a linear DNA fragment (SEQ ID NO: 30) were prepared by following primers, an E. coli variant has deleted wza, wzb, wzc, wcaABCDEF, gmd, wcaGHI, manC, manB, wcaJ, wzx and wcaKL genes was prepared and named as DEB13.

Primer AA:

(SEQ ID NO: 21)

5′-gttatgaaatccctggcgt-3′
Primer AB:

(SEQ ID NO: 22)

5′-ggctttatagaggagaacgcat-3′
Primer BA:

(SEQ ID NO: 23)

5′-atgcgttctcctctataaagccccgcttatcaaggttactgac-3′
Primer BB:

(SEQ ID NO: 24)

5′-attaatttcgataagccagatgatcaacctaaagaaactcctaa-3′
Primer CA:

(SEQ ID NO: 25)

5′-gagtcgacctgcaggcatgcccattgtgtgttagcacca-3′
Primer CB:

(SEQ ID NO: 26)

5′-gctgatttcgatctcgaca-3′

### 1-4. Preparation of DEB21 E. coli Variant Deleted with csg Operon and fim Operon

An E. coli variant that has deleted csg operon and fim operon was prepared by successively using the methods of Example 1-1 and 1-2, and was named DEB21.

### 1-5. Preparation of DEB22 E. coli Variant Deleted with csg Operon and wca Gene Clusters

An E. coli variant with csg operon and wca gene clusters deleted was prepared by successively using the methods of Example 1-1 and 1-3, and thus was named DEB22.

### 1-6. Preparation of DEB23 E. coli Variants Deleted with fim Operon and wca Gene Clusters

An E. coli variant with deleted fim operon and wca gene clusters was prepared by using the methods of Examples 1-2 and 1-3 successively, and thus were named DEB23.

### 1-7. Preparation of DEB31 E. coli Variant that has Deleted All of the csg Operon, fim Operon and wca Gene Clusters

An E. coli variant with csg operon, fim operon and wca gene clusters deleted were prepared by using the methods of Examples 1-1, 1-2 and 1-3, successively. The variant was named DEB31 and was deposited with the Korean Collection for Type Cultures (KCTC) on Nov. 13, 2002 (Deposit No. KCTC 10374BP).

### Example 2

### Confirmation of Inhibition of Biofilm Formation

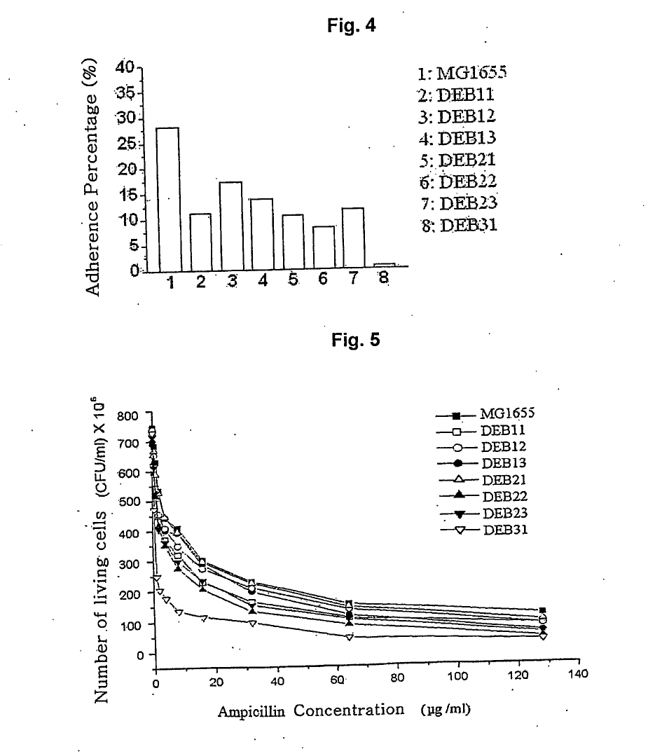

In order to determine any inhibiting effect of the E. coli variants obtained from Examples 1-1 to 1-7, in comparison with the wild-type cell MG1655, the adherence percentages of the variants and the parent cell were measured using the above-mentioned method developed by Dorel. The results were shown in FIG. 4.
Adherence Percentage (%)=(Number of attached cells)/(Number of total cells)×100

As a result, even the variants with one gene deleted, DEB11, DEB12 and DEB13, showed a significant decrease in the number of microorganisms that adhered to the wall. In particular, about 15% of adherence of the DEB11, the one which has curli-related genes deleted, has decreased.

Moreover, when more target regions were deleted, the number of adhered cells was even more remarkably decreased. When all target regions were deleted from the E. coli genome, only about 6% of the E. coli DEB31 was adhered to the well (FIG. 4).

### Example 3

### Measuring the Antibiotics Susceptibility of the Variants

The E. coli variants that have a remarkable inhibiting effect of biofilm formation after deleting biofilm-related genes, confirmed in Example 2, were tested in order to determine whether the reduced biofilm formation substantially increases antibiotics susceptibility.

After incubating the variants in 96-well plates containing 200 μl of LB medium for 24 hours, the serial diluted antibiotics was added to each well at the following concentration ranges: ampicillin, from 0 to 128 μg/ml; streptomycin, from 0 to 64 μg/ml; and rifampicin, from 0 to 256 μg/ml. The cells were incubated for additional 10 hours at 30° C. after antibiotics addition. Then, the number of the living E. coli variants was counted using the standard plate counting method. The results were shown in FIGS. 5, 6 and 7.

As shown in FIG. 5, in case of ampicillin, the number of living wild-type MG1655 selected at the standard concentration of 50 μg/ml was the same as that of living E. coli DEB31 selected at the concentration of 2 μg/ml. In other words, the susceptibility of the biofilm-deficient E. coli variants to ampicillin was increased 25-fold, and the same selection effect could be obtained with the amount of ampicillin that is 25 times reduced.

As shown in FIG. 6, in case of streptomycin, the number of living wild-type MG1655 selected at the standard concentration of 30 μg/ml was the same as that of living E. coli DEB31 selected at the concentration of 8 μg/ml. In other words, the susceptibility of the biofilm-deficient E. coli variants to streptomycin was increased 4-fold, and the same selection effect could be obtained with the amount of streptomycin that is 4 times reduced.

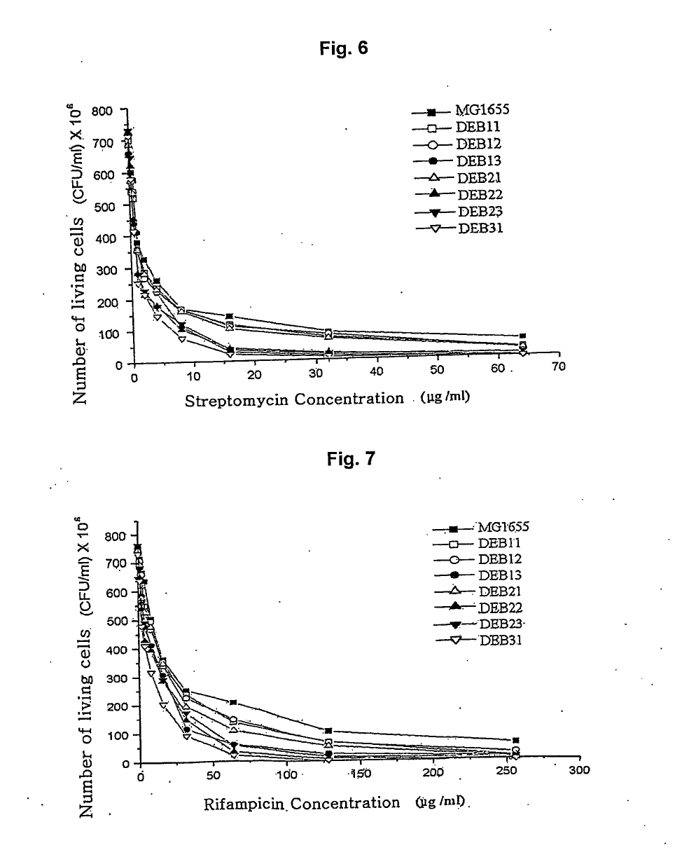

The similar results were shown with rifampicin. As shown in FIG. 7, the number of living wild-type E. coli MG1655 selected at the standard lo concentration of 150 μg/ml was the same as that of living E. coli DEB31 variant selected at the concentration of 32 μg/ml. In other words, the E. coli variants of the present invention were confirmed to have about 5 times increased susceptibility to rifampicin.

### Example 4

### Measuring the Solute Diffusion Efficiency of the Variants

The E. coli variants that were confirmed to have remarkably reduced biofilm formation by deleting biofilm-related genes in Example 2 were tested to confirm whether the inhibition of the biofilm formation substantially increased the solute diffusion efficiency.

A lipase expression vector, pHOPE (Ahn J. H., J. Bacteriol, 181, 1847, 1999) and a lipase ABC transporter expression vector, pABC-ACYC (Ahn J. H., J. Bacteriol, 181, 1847, 1999) were transferred into the cells by using the standard electroporation method. The variants were cultured in a 250 ml Erlenmeyer flask containing 50 ml of LB medium. A part of the culture (1 ml) was harvested with time interval and was centrifuged at 13,000 rpm for 10 minutes. The supernatant (200 μl) was mixed with 3 ml of 100 μM p-nitrophenyl palmitate before incubating for 10 minutes at 45′ . Then, the activity of lipase was detected by measuring the absorbance at 405 nm. One unit of lipase activity was defined as the amount of enzyme necessary to degrade 1 μmol of p-nitrophenyl paimitate per min.

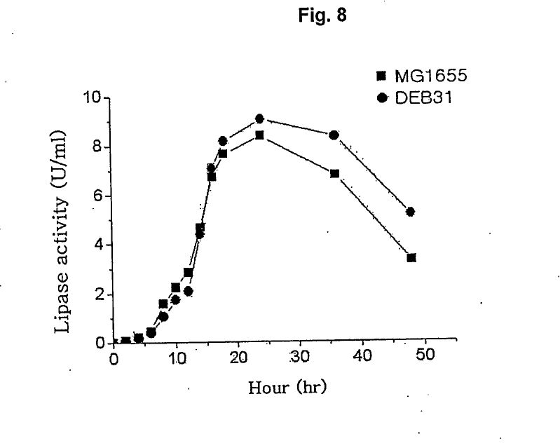

The results are shown in FIG. 8. As shown in FIG. 8, after 24 hours of the incubation, the amount of secreted lipase in the medium was maximized, exhibiting 11% increased activity of lipase.

### Example 5

### Measuring the Transformation Efficiency of the Variants

The E. coli variants that were confirmed in Example 2 that the biofilm formation was remarkably reduced by deleting biofilm-related genes were tested to determine whether the reduction of the biofilm formation substantially increased the transformation efficiency.

The plasmid vector, pUC19 was transferred to wild-type MG1655 and biofilm formation-inhibited DEB31 by using the calcium chloride-heat shock transformation method (Molchanova, E. S., Genetika, 19, 375, 1983). The transformants were spread on LB plate containing ampicillin. Then, they were incubated at 37° C. for 16 hours to determine the number of surviving variants. The result is shown in Table 1.

As shown in Table 1, the transformation efficiency of the biofilm formation-inhibited variants was 20-fold higher than that of the wild-type.

TABLE 1
Transformation Efficiency of Biofilm Formation-Inhibited Variant

| Amount (ng) of Transformed | Transformation Efficiency (CFU/μg) | |
|---|---|---|
| pUC19 | MG1655 | DEB31 |
| 1 | 3.3 × 10⁵ | 7.4 × 10⁶ |
| 10 | 3.4 × 10⁵ | 8.1 × 10⁶ |
| 100 | 3.6 × 10⁵ | 8.1 × 10⁶ |

### INDUSTRIAL APPLICABILITY

As mentioned above, the present invention relates to Escherichia coli variants that have increased antibiotics susceptibility by deleting biofilm-related genes such that a lesser amount of antibiotics are needed to have the same selection efficiency and the surviving rate of strains other than the desirable variants can be decreased. Therefore, the present invention can be useful in the biological product industry. Moreover, the solute diffusion efficiency can be increased due to the inhibition of biofilm formation, the amount of secreted products could be increased. Furthermore, the increased transformation efficiency due to inhibition of biofilm formation also makes the production of useful materials easier. In addition, it reduces the cost of equipments that are used in producing biological products by slowing down the aging of the equipment due to biofilm formation.

1. A linear DNA fragment for deletion of a microbial genomic region comprising:
homology arms A and C of about 500 base pairs at both ends for λ-Red recombination;
a selectable marker, an I-SceI restriction enzyme recognition site (SEQ ID NO: 32) and sacB gene (SEQ ID NO: 31); and
a homology arm B for homologous recombination that can be connected to either the homology arm A or C,
wherein the selectable marker, the I-SceI restriction enzyme recognition site, the sacB gene and the homology arm B are placed between the homology arms A and C,
the homology arms A and C are each homologous to a genomic region of about 500 base pairs that is connected to one of the both ends of the gene region of the microorganism to be deleted, and
the homology arm B is homologous to the microorganism's gene region of about 500 base pairs that is connected to one of the target deletion gene regions of the microorganism that is homologous to the homology arms A and C.

2. The linear DNA fragment of claim 1 having a base sequence of SEQ ID NO: 10, 20 or 30.

3. E. coli variants characterized in that biofilm formation is inhibited by deletion of at least one of csg operon, fim operon, and wca gene clusters, which are biofilm-related genes, by the λ-Red recombination and the homologous recombination using the linear DNA fragment of claim 1.

4. The E. coli variants of claim 3, wherein the λ-Red recombination and the homologous recombination occurs using at least one linear DNA fragment selected from the group consisting of DNA fragments having base sequences of SEQ ID NO: 10, 20 and 30.

5. The E. coli variants of claim 4, wherein the λ-Red recombination and the homologous recombination are repeated using at least two linear DNA fragments selected from the group consisting of DNA fragments having base sequences of SEQ ID NO: 10, 20 and 30.

6. The E. coli variant of claim 5, wherein the λ-Red recombination and the homologous recombination are repeated using all of the linear DNA fragments having base sequences of SEQ ID NO: 10, 20 and 30.

7. The E. coli variant of claim 6, wherein the variant is DEB 31 (Deposit No. KCTC 10374BP).

8. A method for preparing an E. coli variant with a deleted particular genomic region comprising the steps of:
1) preparing the linear DNA fragment of claim 1;
2) inserting the linear DNA fragment into a microorganism and replacing a genomic region to be deleted with the linear DNA fragment by λ-Red recombination of the homology arms A and C with their homologous genomic regions on the chromosome, followed by selecting the transformed microorganism by incubating in a medium containing antibiotics corresponding to the selectable marker; and
3) introducing an I-SceI restriction enzyme expression vector into the selected microorganisms, thereby deleting the selectable marker and sacB genes by homologous recombination between the two homology arm Bs, followed by incubating in a medium containing sucrose to select the markerless deletion variants that does not have any foreign marker.

9. The method of claim 8, wherein step 2) and step 3) are repeated to delete at least one genomic region.

10. The method of claim 8, wherein at least one of the csg operon, fim operon, and wca gene clusters is deleted.

11. The method of claim 10, wherein the csg operon is deleted by using the homology arms A, B and C having SEQ ID NO: 7, 8 and 9, respectively; the fim operon is deleted by using the homology arms A, B and C having SEQ ID NO: 17, 18 and 19, respectively; or the wca gene clusters are deleted by using the homology arms A, B and C having SEQ ID NO: 27, 28 and 29, respectively.

12. The method of claim 11, wherein at least two genes selected from the group consisting of csg operon, fim operon and wca gene clusters are deleted.

13. The method of claim 12, wherein all of the csg operon, fim operon, and wca gene clusters are deleted.

14. The method of claim 8, wherein at least one of the csg operon, fim operon, and wca gene clusters is deleted.

15. The method of claim 14, wherein the csg operon is deleted by using the homology arms A, B and C having SEQ ID NO: 7, 8 and 9, respectively; the fim operon is deleted by using the homology arms A, B and C having SEQ ID NO: 17, 18 and 19, respectively; or the wca gene clusters are deleted by using the homology arms A, B and C having SEQ ID NO: 27, 28 and 29, respectively.

16. The method of claim 15, wherein at least two genes selected from the group consisting of csg operon, fim operon and wca gene clusters are deleted.

17. The method of claim 16, wherein all of the csg operon, fim operon, and wca gene clusters are deleted.


SEQUENCE LISTING (34 sequences)

SEQ ID NO: 1 - DNA, 21 bp, Artificial Sequence
  primer AA for csg operon deletion
ggtgactggaaactggtgtta

SEQ ID NO: 2 - DNA, 21 bp, Artificial Sequence
  primer AB for csg operon deletion
attatcggttatgaaagcaac

SEQ ID NO: 3 - DNA, 44 bp, Artificial Sequence
  primer BA for csg operon deletion
gttgctttcataaccgataataccattattcctgaagtcactct

SEQ ID NO: 4 - DNA, 44 bp, Artificial Sequence
  primer BB for csg operon deletion
attaatttcgataagccagatcagttcatttctacgggtgatga

SEQ ID NO: 5 - DNA, 39 bp, Artificial Sequence
  primer CA for csg operon deletion
gagtcgacctgcaggcatgcattgcagcaatcgtattct

SEQ ID NO: 6 - DNA, 21 bp, Artificial Sequence
  primer CB for csg operon deletion
taaaggttatctgactggaaa

SEQ ID NO: 7 - DNA, 612 bp, Artificial Sequence
  Homology Arm ′A′ for csg operon deletion
ggtgactggaaactggtgttaccttcgctggcgcttgggctgatatttggttacgcaatgaaaaacagtggcctgtggctggcggcgcgtagtgcaaagacggcgcaccgtgagcaggaaatcaaaaataaagcgtgaggggcactcacgctttcgcttaaacagtaaaatgccggatgataattccggcttttttatctgtcaggattccggtggaaccgacatatggcggtatttcaccagaatgtcattctgccgttctgctttattttgcaaatcccacagaccacggtcgataccatcattaatcaggaaaatgacccctgtttcgatagccgacatcaggcacagcataacaggttcgttcgaggtgtaacccacttccccttcaagcaagcgctggtagtcaataaagcggaaaaccccggcctgaacttcataggaaagtatcgtcttactggtgttcaccgaagaaaggatctcgccggtactcacattgacgacgcgcaggttcacggcaatctgatcgagctggtattgcgtgtcggcaccgatgccaaaatatcttgccccaaccccgccagatttgacgttgctttcataaccgataat

SEQ ID NO: 8 - DNA, 584 bp, Artificial Sequence
  Homology Arm ′B′ for csg operon deletion
gttgctttcataaccgataataccattattcctgaagtcactcttactcaatcttgtctgtgcagagtacaaatattgtccctgcgcgaaggcagttcagggcaaagtcagacgaagcaagaaaagaccctttcattgcctgctaatcaacccattgctttgacgaagttgagtttaaatatttccccggacgatcgggtgaaaatagttgttactgtttctgatggacagtcacttcatttatcacaacaatggccgccctcttcagaaaagtcttaatttgttgaaatatcgagcataagatgaatctggagagaatggtctgctgcgaatcagccaacctgaaagtatggataacacaaccctcaaggatgactaatcattgaggaaatagaataaatgttcagaccttttttaaactctcttatgctcggcagtttgttttttccttttattgccattgctggaagcaccgtgcaagggggcgtgatccatttttatggccaaattgtggaaccggcatgtgacgtcagcacccagtcatcacccgtagaaatgaactgatctggcttatcgaaattaat

SEQ ID NO: 9 - DNA, 559 bp, Artificial Sequence
  Homology Arm ′C′ for csg operon deletion
gagtcgacctgcaggcatgcattgcagcaatcgtattctccggtagcgctctggcaggtgttgttcctcagtacggcggcggcggtaaccacggtggtggcggtaataatagcggcccaaattctgagctgaacatttaccagtacggtggcggtaactctgcacttgctctgcaaactgatgcccgtaactctgacttgactattacccagcatggcggcggtaatggtgcagatgttggtcagggctcagatgacagctcaatcgatctgacccaacgtggcttcggtaacagcgctactcttgatcagtggaacggcaaaaattctgaaatgacggttaaacagttcggtggtggcaacggtgctgcagttgaccagactgcatctaactcctccgtcaacgtgactcaggttggctttggtaacaacgcgaccgctcatcagtactaatacatcatttgtattacagaaacagggcgcaagccctgttttttttcgggagaagaatatgaatacgttattactccttgcggcactttccagtcagataaccttta

SEQ ID NO: 10 - DNA, 4677 bp, Artificial Sequence
  DNA fragment for csg operon deletion
ggtgactggaaactggtgttaccttcgctggcgcttgggctgatatttggttacgcaatgaaaaacagtggcctgtggctggcggcgcgtagtgcaaagacggcgcaccgtgagcaggaaatcaaaaataaagcgtgaggggcactcacgctttcgcttaaacagtaaaatgccggatgataattccggcttttttatctgtcaggattccggtggaaccgacatatggcggtatttcaccagaatgtcattctgccgttctgctttattttgcaaatcccacagaccacggtcgataccatcattaatcaggaaaatgacccctgtttcgatagccgacatcaggcacagcataacaggttcgttcgaggtgtaacccacttccccttcaagcaagcgctggtagtcaataaagcggaaaaccccggcctgaacttcataggaaagtatcgtcttactggtgttcaccgaagaaaggatctcgccggtactcacattgacgacgcgcaggttcacggcaatctgatcgagctggtattgcgtgtcggcaccgatgccaaaatatcttgccccaaccccgccagatttgacgttgctttcataaccgataatgatctggcttatcgaaattaatacgactcactatagggagaccggaattcgagctcggtaccttttggcgaaaatgagacgttgatcggcacgtaagaggttccagctttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccgaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcctcactgattaagcattggtaactgtcagatccggggaatttatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatccgctagggataacagggtaatatagatcctctagagtcgacctgcaggcatgcgttgctttcataaccgataataccattattcctgaagtcactcttactcaatcttgtctgtgcagagtacaaatattgtccctgcgcgaaggcagttcagggcaaagtcagacgaagcaagaaaagaccctttcattgcctgctaatcaacccattgctttgacgaagttgagtttaaatatttccccggacgatcgggtgaaaatagttgttactgtttctgatggacagtcacttcatttatcacaacaatggccgccctcttcagaaaagtcttaatttgttgaaatatcgagcataagatgaatctggagagaatggtctgctgcgaatcagccaacctgaaagtatggataacacaaccctcaaggatgactaatcattgaggaaatagaataaatgttcagaccttttttaaactctcttatgctcggcagtttgttttttccttttattgccattgctggaagcaccgtgcaagggggcgtgatccatttttatggccaaattgtggaaccggcatgtgacgtcagcacccagtcatcacccgtagaaatgaactgatctggcttatcgaaattaatgagtcgacctgcaggcatgcattgcagcaatcgtattctccggtagcgctctggcaggtgttgttcctcagtacggcggcggcggtaaccacggtggtggcggtaataatagcggcccaaattctgagctgaacatttaccagtacggtggcggtaactctgcacttgctctgcaaactgatgcccgtaactctgacttgactattacccagcatggcggcggtaatggtgcagatgttggtcagggctcagatgacagctcaatcgatctgacccaacgtggcttcggtaacagcgctactcttgatcagtggaacggcaaaaattctgaaatgacggttaaacagttcggtggtggcaacggtgctgcagttgaccagactgcatctaactcctccgtcaacgtgactcaggttggctttggtaacaacgcgaccgctcatcagtactaatacatcatttgtattacagaaacagggcgcaagccctgttttttttcgggagaagaatatgaatacgttattactccttgcggcactttccagtcagataaccttta

SEQ ID NO: 11 - DNA, 19 bp, Artificial Sequence
  primer AA for fim operon deletion
caatctcatggcgtaagct

SEQ ID NO: 12 - DNA, 20 bp, Artificial Sequence
  primer AB for fim operon deletion
gatttcactatgggtcagga

SEQ ID NO: 13 - DNA, 37 bp, Artificial Sequence
  primer BA for fim operon deletion
cctgacccatagtgaaatcgtctgggattaacggcaa

SEQ ID NO: 14 - DNA, 41 bp, Artificial Sequence
  primer BB for fim operon deletion
attaatttcgataagccagatctagatccagcaactggtca

SEQ ID NO: 15 - DNA, 39 bp, Artificial Sequence
  primer CA for fim operon deletion
gagtcgacctgcaggcatgcccggaaaccattacagact

SEQ ID NO: 16 - DNA, 18 bp, Artificial Sequence
  primer CB for fim operon deletion
ccgtgttattcgctggaa

SEQ ID NO: 17 - DNA, 560 bp, Artificial Sequence
  Homology Arm ′A′ for fim operon deletion
caatctcatggcgtaagctgacgaatcagcaggaataatcgctagggacctaagaattagcatgataatagccactaagaaattactgcgctccatgaaatagccattttgtggcaaatggagttgactaataatgtcatatgtgagacggctagttgaacgaatattaaattttgctgaattttttatgttgattttacttgttacagaacatatcacatgatatatagataagattagttgcattaatgatgagggttattattagattcgtatccgattgataaatatataaaggtacatagcatgcaagagcatggcgtttgtatggcaacgttattataattaacagttgctactccatttaagttcactcagaagaactggtccacttacgttagttattaagcaaacgttcgcttttataaacataatcaggataaaaatgttggattattgctaacccagcacagctagtgcgcgtctgtaattataagggaaaaacgatgaagaataaggctgataacaaaaaaaggaacttcctgacccatagtgaaatc

SEQ ID NO: 18 - DNA, 605 bp, Artificial Sequence
  Homology Arm ′B′ for fim operon deletion
cctgacccatagtgaaatcgtctgggattaacggcaaattatgcacgtaccggagggcaggtgactgcagggaatgtgcaatcgattattggcgtgacttttgtttatcaataaagaaatcacaggacattgctaatgctggtacgcaatattacctgaagctaaaaacctgcacgttagccctttgtaggccagataagacgcgtcagcgtcgcatctggcataaacaaagcgcactttgctggtctgttcccctcaccctaaccctctccccggaggggcgaggggactgtccgggcacatttttagactttgtcatcagtctgagcctgccattggcaggctctggtgtccttttacgctaccatgctaataatcagcacaataatcagcccaaccacggagttgaccagctccagcagaccccaggttttcaacgtgtcttttactgacaggtcaaagtaacctttgaacaaccagaaagatgcatcgttaatgtgggtgagggtgttggaacccgcagccgtcgccagtaccagcagcgccggattcacgccaaccagctgaccagttgctggatctagatctggcttatcgaaattaat

SEQ ID NO: 19 - DNA, 588 bp, Artificial Sequence
  Homology Arm ′C′ for fim operon deletion
gagtcgacctgcaggcatgcccggaaaccattacagactatgtcacactgcaacgaggctcggcttatggcggcgtgttatctaatttttccgggaccgtaaaatatagtggcagtagctatccatttcctaccaccagcgaaacgccgcgcgttgtttataattcgagaacggataagccgtggccggtggcgctttatttgacgcctgtgagcagtgcgggcggggtggcgattaaagctggctcattaattgccgtgcttattttgcgacagaccaacaactataacagcgatgatttccagtttgtgtggaatatttacgccaataatgatgtggtggtgcctactggcggctgcgatgtttctgctcgtgatgtcaccgttactctgccggactaccctggttcagtgccaattcctcttaccgtttattgtgcgaaaagccaaaacctggggtattacctctccggcacaaccgcagatgcgggcaactcgattttcaccaataccgcgtcgttttcacctgcacagggcgtcggcgtacagttgacgcgcaacggtacgattattccagcgaataacacgg

SEQ ID NO: 20 - DNA, 4675 bp, Artificial Sequence
  DNA fragment for fim operon deletion
caatctcatggcgtaagctgacgaatcagcaggaataatcgctagggacctaagaattagcatgataatagccactaagaaattactgcgctccatgaaatagccattttgtggcaaatggagttgactaataatgtcatatgtgagacggctagttgaacgaatattaaattttgctgaattttttatgttgattttacttgttacagaacatatcacatgatatatagataagattagttgcattaatgatgagggttattattagattcgtatccgattgataaatatataaaggtacatagcatgcaagagcatggcgtttgtatggcaacgttattataattaacagttgctactccatttaagttcactcagaagaactggtccacttacgttagttattaagcaaacgttcgcttttataaacataatcaggataaaaatgttggattattgctaacccagcacagctagtgcgcgtctgtaattataagggaaaaacgatgaagaataaggctgataacaaaaaaaggaacttcctgacccatagtgaaatccctgacccatagtgaaatcgtctgggattaacggcaaattatgcacgtaccggagggcaggtgactgcagggaatgtgcaatcgattattggcgtgacttttgtttatcaataaagaaatcacaggacattgctaatgctggtacgcaatattacctgaagctaaaaacctgcacgttagccctttgtaggccagataagacgcgtcagcgtcgcatctggcataaacaaagcgcactttgctggtctgttcccctcaccctaaccctctccccggaggggcgaggggactgtccgggcacatttttagactttgtcatcagtctgagcctgccattggcaggctctggtgtccttttacgctaccatgctaataatcagcacaataatcagcccaaccacggagttgaccagctccagcagaccccaggttttcaacgtgtcttttactgacaggtcaaagtaacctttgaacaaccagaaagatgcatcgttaatgtgggtgagggtgttggaacccgcagccgtcgccagtaccagcagcgccggattcacgccaaccagctgaccagttgctggatctagatctggcttatcgaaattaatgatctggcttatcgaaattaatacgactcactatagggagaccggaattcgagctcggtaccttttggcgaaaatgagacgttgatcggcacgtaagaggttccagctttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccgaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcctcactgattaagcattggtaactgtcagatccggggaatttatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatccgctagggataacagggtaatatagatcctctagagtcgacctgcaggcatgcgagtcgacctgcaggcatgcccggaaaccattacagactatgtcacactgcaacgaggctcggcttatggcggcgtgttatctaatttttccgggaccgtaaaatatagtggcagtagctatccatttcctaccaccagcgaaacgccgcgcgttgtttataattcgagaacggataagccgtggccggtggcgctttatttgacgcctgtgagcagtgcgggcggggtggcgattaaagctggctcattaattgccgtgcttattttgcgacagaccaacaactataacagcgatgatttccagtttgtgtggaatatttacgccaataatgatgtggtggtgcctactggcggctgcgatgtttctgctcgtgatgtcaccgttactctgccggactaccctggttcagtgccaattcctcttaccgtttattgtgcgaaaagccaaaacctggggtattacctctccggcacaaccgcagatgcgggcaactcgattttcaccaataccgcgtcgttttcacctgcacagggcgtcggcgtacagttgacgcgcaacggtacgattattccagcgaataacacgg

SEQ ID NO: 21 - DNA, 19 bp, Artificial Sequence
  primer AA for wca operon deletion
gttatgaaatccctggcgt

SEQ ID NO: 22 - DNA, 22 bp, Artificial Sequence
  primer AB for wca operon deletion
ggctttatagaggagaacgcat

SEQ ID NO: 23 - DNA, 43 bp, Artificial Sequence
  primer BA for wca operon deletion
atgcgttctcctctataaagccccgcttatcaaggttactgac

SEQ ID NO: 24 - DNA, 44 bp, Artificial Sequence
  primer BB for wca operon deletion
attaatttcgataagccagatcgtcaacctaaagaaactcctaa

SEQ ID NO: 25 - DNA, 39 bp, Artificial Sequence
  primer CA for wca operon deletion
gagtcgacctgcaggcatgcccattgtgtgttagcacca

SEQ ID NO: 26 - DNA, 19 bp, Artificial Sequence
  primer CB for wca operon deletion
gctgatttcgatctcgaca

SEQ ID NO: 27 - DNA, 542 bp, Artificial Sequence
  Homology Arm ′A′ for wca operon deletion
gttatgaaatccctggcgtaagatggcgtaattagcgtggctaacggtcaggttatcgatgatcaggttgcgcatgacccgtttgtttttgccgccgatataaatctgcgtcaccgggccaaagccgctcatagtcagccctttgatggtgcagtcagaaccacgcacatccagggtgatgttatgcatactgccgccatcctcccctgtcacctggctgccgtcctgtaagacaaatcgccctctgccgttgccgcgcaagcttccaagaatgtgtaacgttttaccgggagggataaagatgccggtgttgatattgtcacaaaccaatccggcaggcacgacgactgtttgcccttcgctgaaggcttgtttaaatgaggcgatccagtcgtgtgggttgtagtcgttaatgttaacgctttgtcgggcgggaagcgcgcgggcgaaaggggtatggaggaaggcaagcgccgagcttgccgtcaggaacgtgcgtcgggagagttttttaaatggcatgcgttctcctctataaagcc

SEQ ID NO: 28 - DNA, 562 bp, Artificial Sequence
  Homology Arm ′B′ for wca operon deletion
atgcgttctcctctataaagccccgcttatcaaggttactgacaccaataatggcatcaatttcattttggatttcatcattgtttatttatcactttggcagagtaattatcctgtgcactattaatagcaatgtcgccatgcacatttaccttgcagttaattgaataaaaatttaactggcatcagtcctaaaaaaattgatttcatccgcaggctattgacagaataattcagactggtctttcaggcatccagacacgctaccgcccctggctttttagctaccaatacactgatttagtttaatttttcacaccctctcagcatgcagtcgttgatgagaaagggttattacggaaattaacttccgaatataaggtgacattatggtaattgaatattggctttccaataatgcaggaggaagtgttacagctaacggaatagcaggcaagataacaattcggtaattggctatttttaagaattatattaagtgtcattcaatatggtttttaggagtttctttaggttgacgatctggcttatcgaaattaat

SEQ ID NO: 29 - DNA, 553 bp, Artificial Sequence
  Homology Arm ′C′ for wca operon deletion
gagtcgacctgcaggcatgcccattgtgtgttagcaccacgttgcgccagtcagcggtgtcggtcaggccacctgcggcgttgatggcgtcgagaatagtcagtggcacgttggtgatcgcctgttgaccggatttattcacctgaccggagatataggccttttgtgagcggaaggcggcgatattaacgtccacctgcgggtcagcgatgtacgtcgctaagcgcccggtaatatcactgcggatttcagcgagcgttttcccgactacgtggaccttgccgatatacgggtaaaacatagtgccgtcaggctgtacccagttgccggtgtcgctggagctgcggtactgaccggctggcgtggtgagttccgggtgatcccagacggtgacattaagaacgtcccccggcccgacgcgatactggtaattcgcgatctcactttccagcgtcatattggggcgcgctacattcgggcgtgggcgtaattggtcaatcaggcgcggggtcagcggataaacattcaccattttgtcgagatcgaaatcagc

SEQ ID NO: 30 - DNA, 4579 bp, Artificial Sequence
  DNA fragment for wca operon deletion
gttatgaaatccctggcgtaagatggcgtaattagcgtggctaacggtcaggttatcgatgatcaggttgcgcatgacccgtttgtttttgccgccgatataaatctgcgtcaccgggccaaagccgctcatagtcagccctttgatggtgcagtcagaaccacgcacatccagggtgatgttatgcatactgccgccatcctcccctgtcacctggctgccgtcctgtaagacaaatcgccctctgccgttgccgcgcaagcttccaagaatgtgtaacgttttaccgggagggataaagatgccggtgttgatattgtcacaaaccaatccggcaggcacgacgactgtttgcccttcgctgaaggcttgtttaaatgaggcgatccagtcgtgtgggttgtagtcgttaatgttaacgctttgtcgggcgggaagcgcgcgggcgaaaggggtatggaggaaggcaagcgccgagcttgccgtcaggaacgtgcgtcgggagagttttttaaatggcatgcgttctcctctataaagccatgcgttctcctctataaagccccgcttatcaaggttactgacaccaataatggcatcaatttcattttggatttcatcattgtttatttatcactttggcagagtaattatcctgtgcactattaatagcaatgtcgccatgcacatttaccttgcagttaattgaataaaaatttaactggcatcagtcctaaaaaaattgatttcatccgcaggctattgacagaataattcagactggtctttcaggcatccagacacgctaccgcccctggctttttagctaccaatacactgatttagtttaatttttcacaccctctcagcatgcagtcgttgatgagaaagggttattacggaaattaacttccgaatataaggtgacattatggtaattgaatattggctttccaataatgcaggaggaagtgttacagctaacggaatagcaggcaagataacaattcggtaattggctatttttaagaattatattaagtgtcattcaatatggtttttaggagtttctttaggttgacgatctggcttatcgaaattaatgatctggcttatcgaaattaatacgactcactatagggagaccggaattcgagctcggtaccttttggcgaaaatgagacgttgatcggcacgtaagaggttccagctttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccgaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcctcactgattaagcattggtaactgtcagatccggggaatttatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatccgctagggataacagggtaatatagatcctctagagtcgacctgcaggcatgcgagtcgacctgcaggcatgcccattgtgtgttagcaccacgttgcgccagtcagcggtgtcggtcaggccacctgcggcgttgatggcgtcgagaatagtcagtggcacgttggtgatcgcctgttgaccggatttattcacctgaccggagatataggccttttgtgagcggaaggcggcgatattaacgtccacctgcgggtcagcgatgtacgtcgctaagcgcccggtaatatcactgcggatttcagcgagcgttttcccgactacgtggaccttgccgatatacgggtaaaacatagtgccgtcaggctgtacccagttgccggtgtcgctggagctgcggtactgaccggctggcgtggtgagttccgggtgatcccagacggtgacattaagaacgtcccccggcccgacgcgatactggtaattcgcgatctcactttccagcgtcatattggggcgcgctacattcgggcgtgggcgtaattggtcaatcaggcgcggggtcagcggataaacattcaccattttgtcgagatcgaaatcagc

SEQ ID NO: 31 - DNA, 1429 bp, Artificial Sequence
  SacB gene
atgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtatctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataa

SEQ ID NO: 32 - DNA, 18 bp, Artificial Sequence
  I-SceI restriction site
tagggataacagggtaat

SEQ ID NO: 33 - DNA, 4598 bp, Artificial Sequence
  pST98AS vector
atcgatgggtggttaactcgacatcttggttaccgtgaagttaccatcacggaaaaaggttatgctgcttttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaaatgcatcaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaacaacgtgttaaccacctgggtaacctggtaatcacctggggcgcccagactttcaaacaccaagctttcaacaaactggctaacctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaataataagtcgacccgaattggtcgacaagcttgatctggcttatcgaaattaatacgactcactatagggagaccggaattcgagctcggtacccggggatccgctagggataacagggtaatatagatcctctagagtcgacctgcaggcatgcaagcttggcactggctgatcagctagcccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgc

SEQ ID NO: 34 - DNA, 658 bp, Artificial Sequence
  Chloramphenicol resistant gene
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagatgcttaatgaatacaacagtactgcgatgagtggcagggcggggcgtaa